(19) United States
(12) Patent Application Publication
(10) Pub. No.: US 2022/0205999 A1
(43) Pub. Date: Jun. 30, 2022

(54) METHODS FOR DIAGNOSING AND TREATING INFLAMMATORY BOWEL DISEASE IN COMPANION ANIMALS

(71) Applicant: Colorado State University Research Foundation, Fort Collins, CO (US)

(72) Inventors: Steven DOW, Littleton, CO (US); Sirikul SOONTARARAK, Fort Collins, CO (US); Lyndah CHOW, Fort Collins, CO (US)

(21) Appl. No.: 17/600,300

(22) PCT Filed: Apr. 1, 2020
(86) PCT No.: PCT/US20/26074

§ 371 Date: Sep. 30, 2021

Related U.S. Application Data
(60) Provisional application No. 62/827,436, filed on Apr. 1, 2019.

(51) Int. Cl. G01N 33/564; A61K 31/365; A61K 31/4164; A61K 31/43; A61K 31/473; A61K 31/58; A61K 31/7048; A61K 35/741; A61K 38/13; C07K 16/42

(52) U.S. Cl. CPC G01N 33/564; A61K 31/365; A61K 31/4164; A61K 31/43; A61K 31/473; A61K 2035/115; A61K 31/7048; A61K 35/741; A61K 38/13; C07K 16/4233; A61K 31/58

(57) ABSTRACT

The present disclosure relates to methods for diagnosing and treating inflammatory bowel disease (IBD) in companion animals.

### CROSS-REFERENCE TO RELATED APPLICATIONS

This application claims the benefit of U.S. Provisional Application No. 62/827,436, filed Apr. 1, 2019, incorporated herein by reference in its entirety.

### FIELD

The present disclosure relates to methods for diagnosing and treating inflammatory bowel disease (IBD) in companion animals.

### BACKGROUND

Inflammatory bowel disease (IBD) in dogs is characterized by infiltration of lymphocytes and macrophages into the mucosa and submucosa and clinical signs of gastrointestinal (GI) dysfunction (diarrhea, malabsorption, weight loss). Alteration of the gut environment and development of dysbiosis may allow the overgrowth of pathogenic bacteria and induction of intestinal injury and inflammation in IBD. Genetic and environmental factors are also associated with IBD in dogs and humans. Inflammation in IBD in humans is thought to be mediated by both cellular and humoral immune mechanisms. Increasingly, some studies in IBD in humans (e.g., Chrohn's disease, ulcerative colitis) have focused on the role of immune responses targeted to gut bacteria, as opposed to immune responses targeting gut tissues or dietary antigens.

In dogs with IBD, previous studies have documented dysregulation of humoral immunity, principally a reduction in the overall amount of gut IgA produced. For example, decreased production of mucosal IgA has been documented in dogs with IBD, along with increased production of pro-inflammatory cytokines by gut mucosal immune cells (T cells and macrophages). However, the specificity of gut IgA in dogs has not been previously investigated. Increased numbers of plasma cells have also been observed in the lamina propria of dogs with IBD, consistent with local IgG production.

Little is known regarding antibody recognition of gut bacteria in dogs with IBD. It is known that the microbiome is very different in dogs with IBD than in healthy dogs, and that certain phyla predominate in these bacterial populations. However, diagnosis of IBD in companion animals is generally determined based on clinical symptoms and/or endoscopy. What is needed are novel method fors diagnosing and treating inflammatory bowel disease (IBD) in companion animals.

The methods disclosed herein address these and other needs.

### SUMMARY

Disclosed herein are methods for diagnosing and treating inflammatory bowel disease (IBD) in companion animals. To date, there are no commercially available assays for accurately identifying IBD in companion animals, other than expensive and invasive endoscopic procedures. As disclosed herein, the inventors have developed a novel, non-invasive method for diagnosing and treating IBD in companion animals by determining IgG binding to fecal bacteria in a stool sample.

In some aspects, disclosed herein is a method for treating inflammatory bowel disease (IBD) in a companion animal comprising: collecting a fecal sample from the companion animal; incubating the fecal sample with a detecting antibody which specifically binds to an IgG antibody; determining a proportion of fecal bacteria in the fecal sample that are bound by IgG antibody; diagnosing that the animal is susceptible to or suffering from IBD when the proportion of fecal bacteria in the fecal sample that are bound by the IgG antibody is greater than about 60%; and if the animal is susceptible to or suffering from IBD: i) administering to the companion animal an effective amount of a therapeutic agent for treating the IBD, ii) changing the diet of the companion animal, or iii) performing a fecal transfaunation.

In some embodiments, the detecting antibody is conjugated to a fluorescent moiety.

In some embodiments, the proportion of fecal bacteria in the fecal sample that are bound by the IgG antibody is determined by flow cytometry.

In some embodiments, the companion animal is a canine. In some embodiments, the companion animal is a dog. In some embodiments, the companion animal is a feline. In some embodiments, the companion animal is a cat.

In some embodiments, the therapeutic agent is selected from an antibiotic, an immunosuppressive agent, or a probiotic.

In some embodiments, the antibiotic comprises metronidazole, tylosin, or ampicillin.

In some embodiments, the immunosuppressive agent comprises prednisone, prednisolone, budesonide, cyclosporine, mycophenolate, or chlorambucil.

In some aspects, disclosed herein is a method for diagnosing inflammatory bowel disease (IBD) in a companion animal comprising: collecting a fecal sample from the companion animal; incubating the fecal sample with a detecting antibody which specifically binds to an IgG antibody; determining a proportion of fecal bacteria in the fecal sample that are bound by IgG antibody; and diagnosing that the animal is susceptible to or suffering from IBD when the proportion of fecal bacteria in the fecal sample that are bound by the IgG antibody is greater than about 60%.

In some embodiments, the method further comprises administering to the companion animal an effective amount of a therapeutic agent for treating the IBD, changing the diet of the companion animal, or performing a fecal transfaunation, if the animal is susceptible to or suffering from IBD.

### BRIEF DESCRIPTION OF THE DRAWINGS

The accompanying figures, which are incorporated in and constitute a part of this specification, illustrate several aspects described below.

FIG. 1 shows flow cytometry analysis and gating. Fecal bacteria were analyzed based on size and complexity corresponding to bacteria population as well as selective counting of 10⁵ bacteria cells. The percentage of positive fluorescence cells of IgG-binding bacteria and fluorescence intensity was analyzed by comparing to background threshold.

FIGS. 2A-2D show IgG⁺ and IgA⁺ fecal bacteria in healthy dogs and dogs with IBD. In FIG. 2A the percentages of IgG⁺ bacteria are plotted in dogs with IBD versus healthy dogs. In FIG. 2B the amount of IgG bound to each bacterium (MFI) is plotted for the two groups of animals IgA binding percentages and total IgA binding to each bacterium are depicted in FIGS. 2C and 2D, respectively. Data are plotted as mean±SD. Statistical differences were calculated using two-tailed unpaired t-test (in FIGS. 2A-2C) or a Mann-Whitney U test (in FIG. 2D).

FIG. 3 shows Ig-binding fecal bacteria (A) Immunofluorescence staining and imaging of fecal bacteria from a healthy dog (top row) and from a dog with IBD (bottom row). IgA bound to bacteria indicated as green, while IgG⁺ bacteria indicated as red. Bacteria with both bound antibodies show up as yellow images in merged figures. Scale bar indicates 10 μm.

FIGS. 4A-4B show association between IgG and IgA binding to fecal bacteria. Scatter dot plot of percentage (FIG. 4A) of IgA-bound and IgG-bound bacteria and amount of IgG and IgA binding to individual bacteria (MFI) depicted (FIG. 4B). To analyze the degree of association between IgG and IgA binding, linear regression analysis was performed. The percentage of IgA-bound bacteria was significant correlated with the percentage of IgG-bound bacteria (R²=0.45, P=0.001). Also, degree of IgA and IgG binding also showed significant correlation (R²=0.48, P=0.001). Dashed lines depict 95% confidence band.

FIG. 5 shows serum IgG recognition of E. coli isolated from healthy dogs and dogs with IBD. Six separate fecal isolates of E. coli (3 from dogs with IBD and 3 from healthy dogs) were incubated with serum from dogs with IBD (n=20) and healthy dogs (n=9), and IgG binding to the surface of bacteria was quantitated using flow cytometry, as noted in Methods. Scatter plots depicting IgG⁺ bacteria percentages in healthy versus IBD dogs plotted. The percentages of IgG⁺ bacteria were not significantly different between the two groups of animal sera (P=0.29). (†) Indicates E. coli isolates from normal dogs, while (‡) indicates E. coli isolates from dogs with IBD. Data are reported as mean±SD and statistical differences were calculated using one-way ANOVA.

FIGS. 6A-6C show macrophage phagocytosis of fecal bacteria from dogs with IBD versus healthy dogs. FIG. 6A shows that fecal bacteria (PI staining; red) from dogs with IBD and from healthy dogs (n=5 per group) were incubated with primary cultures of canine monocyte-derived macrophages and bacterial uptake was determined using flow cytometry, as described in Methods. Images were obtained using confocal microscopy, with PI stained bacteria visualized as red objects within cultured macrophages. DAPI staining (blue) demonstrates cell nuclei. Similar results were obtained in at least n=3 repeated, independent studies. Box plot comparing the percentage of macrophages containing intracellular bacteria (FIG. 6B) and the relative number of bacteria per macrophage (FIG. 6C), when bacteria from dogs with IBD and healthy control dogs were compared. Statistical differences were calculated using two-tailed unpaired t-tests (FIG. 6B) and by the Mann-Whitney test (FIG. 6C). Scale bar as indicated.

FIG. 7 shows cytokine production by activated macrophages. Canine monocyte-derived macrophages were activated by incubation and phagocytosis of non-viable fecal bacteria obtained from dogs with IBD (n=5) and from healthy normal dogs (n=5), as described in Methods. TNF-α and IL-10 concentrations in media obtained from macrophage cultures 24 hours after bacterial inoculation were measured using commercial canine-specific ELISA. Box plots comparing cytokine concentrations between the 2 groups of fecal bacterial samples are depicted. Statistical differences were calculated using two-tailed unpaired t-tests. The assays were repeated for 3 times, total of 3 different PBMC donors.

FIGS. 8A-8C shows microbiome analysis. IgG^{hi} sorted fecal bacteria from (n=10) dogs with IBD, and non-sorted bacteria (n=10; paired fecal samples from dogs with IBD) and bacteria from healthy control animals (n=10) were analyzed by 16S rRNA sequencing, as described in Methods. FIG. 8A shows species abundance heat map at taxonomic level representing average differences, with 0=no difference, −1 and 1 representing maximum differences. (†) Showing the top 10 taxa abundance. FIG. 8B shows bar graph depicting the relative abundance of 5 major phyla comparing the IgG^{hi} sorted population with non-sorted bacteria, obtained from same dogs with IBD. A significant increased abundance of Actinobacteria phyla was found in IgG^{hi} sorted population. FIG. 8C shows bar graph showing relative abundance comparing between IgG^{hi} sorted and non-sorted bacteria for members of Actinobacteria phyla. The data were reported as mean±SD, and statistical comparisons were calculated using paired t-test (*P≤0.05, **P≤0.01, ***P≤0.001).

FIG. 9 shows relative abundance of 5 major phyla in dogs with IBD and healthy controls. Significant decrease in Bacteroidetes (P=0.02) and increased Proteobacteria (P=0.045) were observed in dogs with IBD. Bar graphs depict relative abundance of 5 phyla, and statistical differences calculated using unpaired t-test (*P≤0.05 **P≤0.01, ***P≤0.001).

FIG. 10 shows receiver operator curves for bacterial IgG assay. To quantify the diagnostic ability of the bacterial IgG assay to discriminate dogs with IBD (n=20) from normal dogs (n=9) based on percentage IgG-binding gut bacteria, ROC curve analysis was performed. Area under the curve (AUC) was reported as 0.92, SD 0.06, P<0.0001.

FIG. 11 shows association of Collinsella and clinical parameters in IBD. Scatter dot plot of % abundance of Collinsella and clinical parameters depicted. Linear regression analysis was performed. The P value as stated in the figures. Dashed lines depict 95% confidence band. CIBDAI; Canine Inflammatory Bowel Disease Activity Index, CCECAI; Canine Chronic Enteropathy Clinical Activity Index.

FIG. 12 shows dot plots comparing the percentages of IgG⁺ bacteria (left panel) and the amounts of IgG bound to each bacterium (MFI) (right panel) when bacteria from dogs with IBD, giardiasis, and healthy control dogs were compared.

### DETAILED DESCRIPTION

Disclosed herein are methods for diagnosing and treating inflammatory bowel disease (IBD) in companion animals. To date, there are no commercially available assays for accurately identifying IBD in companion animals, other than expensive and invasive endoscopic procedures. As disclosed herein, the inventors have developed a novel, non-invasive method for diagnosing and treating IBD in companion animals by determining IgG binding to fecal bacteria in a stool sample.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “composition” is intended to include a combination of active agent and another compound or composition, inert (for example, a detectable agent or label) or active, such as an adjuvant.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “about” as used herein when referring to a measurable value such as an amount, a percentage, and the like, is meant to encompass variations of ±20%, ±10%, ±5%, or ±1% from the measurable value.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. The phrases “concurrent administration”, “administration in combination”, “simultaneous administration” or “administered simultaneously” as used herein, means that the compounds are administered at the same point in time or immediately following one another.

As used herein, the term “companion animal” refers to those animals traditionally kept for companionship or enjoyment, such as for example, dogs, cats, horses, birds, reptiles, mice, rabbits, hamsters, and the like.

### Methods for Treatment and Diagnosis

In some aspects, disclosed herein is a method for treating inflammatory bowel disease (IBD) in a companion animal comprising: collecting a fecal sample from the companion animal; incubating the fecal sample with a detecting antibody which specifically binds to an IgG antibody; determining a proportion of fecal bacteria in the fecal sample that are bound by IgG antibody; diagnosing that the animal is susceptible to or suffering from IBD) when the proportion of fecal bacteria in the fecal sample that are bound by the IgG antibody is greater than about 60%; and if the animal is susceptible to or suffering from IBD: i) administering to the companion animal an effective amount of a therapeutic agent for treating the IBD, ii) changing the diet of the companion animal, or iii) performing a fecal transfaunation.

In some embodiments, the method of treatment, if the animal is susceptible to or suffering from IBD, comprises administering to the companion animal an effective amount of a therapeutic agent for treating IBD. In some embodiments, the method of treatment, if the animal is susceptible to or suffering from IBD, comprises changing the diet of the companion animal. In some embodiments, the method of treatment, if the animal is susceptible to or suffering from IBD, comprises performing a fecal transfaunation.

In some embodiments, the detecting antibody is conjugated to a fluorescent moiety. In some embodiments, the proportion of fecal bacteria in the fecal sample that are bound by the IgG antibody is determined by flow cytometry.

Flow cytometry is an immunoassay of measuring certain chemical and physical properties of cells, include cell-size and the expression of cell-surface and intracellular markers. Immunoassays, in their most simple and direct sense, are binding assays involving binding between antibodies and antigen. Many types and formats of immunoassays are known and are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent moiety, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent moiety is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al, 1999, In: Using Antibodies: A Laboratory Manual, Cold Spring Harbor Laboratory Press, NY; Harlow et al., 1989, in: Antibodies: A Laboratory Manual, Cold Spring Harbor, New York; Houston et al., 1988, Proc. Natl. Acad. Sci. USA 85:5879-5883; Bird et al., 1988, Science 242:423-426). There are five major classes of canine immunoglobulins: IgA, IgD, IgE, IgG and IgM. One skilled in the art would recognize the comparable classes for mouse or human. The heavy chain constant domains that correspond to the different classes of immunoglobulins are called alpha, delta, epsilon, gamma, and mu, respectively.

In some embodiments, the companion animal is a canine. In some embodiments, the companion animal is a dog. In some embodiments, the companion animal is a feline. In some embodiments, the companion animal is a cat.

In some embodiments, the proportion of fecal bacteria in the fecal sample that are bound by the IgG antibody is greater than about 50% (for example, greater than 50%, greater than 51%, greater than 52%, greater than 53%, greater than 54%, greater than 55%, greater than 56%, greater than 57%, greater than 58%, greater than 59%, greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%). In some embodiments, the proportion of fecal bacteria in the fecal sample that are bound by the IgG antibody is greater than about 60%. In some embodiments, the proportion of fecal bacteria in the fecal sample that are bound by the IgG antibody is greater than 60%.

In some embodiments, the therapeutic agent is selected from an antibiotic, an immunosuppressive agent, or a probiotic. In some embodiments, the therapeutic agent is an antibiotic. In some embodiments, the therapeutic agent is an immunosuppressive agent. In some embodiments, the therapeutic agent is a probiotic.

In some embodiments, the antibiotic comprises metronidazole, tylosin, or ampicillin. In some embodiments, the antibiotic is metronidazole. In some embodiments, the antibiotic is tylosin. In some embodiments, the antibiotic is ampicillin.

In some embodiments, the immunosuppressive agent comprises prednisone, prednisolone, budesonide, cyclosporine, mycophenolate, or chlorambucil. In some embodiments, the immunosuppressive agent is prednisone. In some embodiments, the immunosuppressive agent is cyclosporine. In some embodiments, the immunosuppressive agent is mycophenolate. In some embodiments, the immunosuppressive agent is chlorambucil.

In some aspects, disclosed herein is a method for diagnosing IBD in a companion animal comprising: collecting a fecal sample from the companion animal; incubating the fecal sample with a detecting antibody which specifically binds to an IgG antibody; determining a proportion of fecal bacteria in the fecal sample that are bound by IgG antibody; and diagnosing that the animal is susceptible to or suffering from IBD when the proportion of fecal bacteria in the fecal sample that are bound by the IgG antibody is greater than about 60%. The method in the present invention is surprisingly specific for diagnosing IBD in a companion animal, distinguishing from dogs with gut inflammation due to other disorders, including, for example, microbial infections. Further, the method disclosed herein is highly specific for detecting bacteria that are produced locally in the gastrointestinal tract.

In some embodiments, the method further comprises administering to the companion animal an effective amount of a therapeutic agent for treating the IBD or changing the diet of the companion animal, if the animal is susceptible to or suffering from IBD.

In some embodiments, the fecal sample is a fresh sample. In some embodiments, the fecal sample can be a frozen sample.

In some embodiments, the method further comprises a step for processing the fecal sample to produce a fecal bacterial suspension. In some embodiments, the method further comprises a step wherein the fecal bacterial suspension is centrifuged to obtain a bacterial pellet. In some embodiments, the bacterial pellet is resuspended and incubated with a detecting antibody.

In some embodiments, the detecting antibody is an anti-dog IgG antibody. In some embodiments, the detecting antibody is a rabbit anti-dog IgG antibody (for example, the Alexa Fluor® AffiniPure rabbit anti-dog IgG from Jackson ImmunoResearch Laboratories). Additional anti-dog IgG antibodies are known in the art and are available from, for example, BioRad and Bethyl Laboratories. In some embodiments, the detecting antibody is an anti-cat IgG antibody. Anti-cat IgG antibodies are known in the art and are available from, for example, Bethyl Laboratories.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Humoral Immune Response Against Gut Bacteria in Dogs with Inflammatory Bowel Disease (IBD)

To date, there are no commercially available assays for accurately identifying IBD in companion animals, other than expensive and invasive endoscopic procedures. In this example, the inventors have developed a novel, non-invasive method for diagnosing and treating IBD in companion animals by determining IgG binding to fecal bacteria in a fresh or frozen stool sample. Use of this test can avoid the need for an expensive and invasive endoscopic biopsy for an IBD diagnosis, and can also be used to monitor the effectiveness of treatment, since the test provides a quantitative assessment of the intensity of the immune response against commensal bacteria in dogs with IBD.

### Material and Methods

### Study Populations

A prospective observational study was conducted at the Colorado State University Veterinary Teaching Hospital (CSU-VTH), approved by the Clinical Review Board (CRB) and the Institutional Animal Care and Use Committee (IACUC) at CSU. Dog owners were informed regarding the study protocol, and consent was obtained before enrollment in the study. A total of 29 dogs, 20 dogs diagnosed with IBD and 9 healthy control dogs, were evaluated.

### IBD Dogs

Twenty dogs with IBD dogs (14 male and 6 female) with persistent signs of gastroenteritis, including vomiting, diarrhea, weight loss, for a minimum 3 weeks were recruited into the study. All dogs with IBD had undergone endoscopy and intestinal biopsy to confirm a diagnosis of IBD and rule out intestinal lymphoma. Most animals had previously undergone and failed food trials including elimination diet, novel protein and/or hydrolyzed protein at least 3 weeks. All dogs had no recent history of receiving immunosuppressive medications and were free from other diseases causing chronic GI dysfunction including hepatic disease, pancreatic insufficiency, metabolic disease parasitic disease and renal disease. German Shepherd dogs were purposely excluded, as this breed is known to be predisposed to defective intestinal IgG and IgA production.

All study dogs with IBD received clinical evaluation including disease activity index evaluation (Canine Inflammatory Bowel Disease Activity Index; CIBDAI and Canine Chronic Enteropathy Clinical Activity Index; CCECAI), CBC, serum chemistry profile, and fecal examination for parasites. The H&E stained intestinal biopsy specimens from dogs with IBD underwent WSAVA histopathologic score evaluation by a board certified veterinary pathologist. Additional tests performed in dogs with IBD included serum folate concentration and serum cobalamin concentration (Gastrointestinal Laboratory, Texas A&M University, TX)

### Clinical Healthy Controls Animals

Nine clinically healthy dogs (4 males and 5 females) that were also age-matched to the IBD dogs were enrolled. These dogs were client owned and were evaluated at the CSU-VTH for a health checkup. Based on history and normal physical examination these animals were judged to not have any clinical signs indicative of gastrointestinal disease, and the animals had no history of immune mediated disease or immune suppressive medication usage. All dogs had complete blood count (CBC) and serum chemistry profiles performed, and all evaluations were within the normal limits.

### Sample Collection

Stool samples and blood samples from all study dogs were collected and stored at 4° C. immediately prior to sample preparation, which occurred within 4 hours of sample collection. Serum samples were stored at −80° C. Stool samples were obtained by spontaneous defecation and/or rectal palpation. Fresh stool samples were processed to generate a fecal bacteria suspension as described previously. Briefly, 0.5 g of stool was homogenized in 24.5 ml of sterile-filtered phosphate buffer saline (PBS; 0.2 μm-filtered) solution using vortexing, then centrifuged at 700× G for 5 minutes. The washed bacteria were collected and stored in 1 ml aliquots at −80° C. until used.

### Flow Cytometry

The fecal bacteria suspension was centrifuged at 10,000× G for 5 minutes to obtain a bacterial pellet, which was washed with PBS once. For measurement of Ig-binding fecal bacteria, the bacterial pellet was resuspended in 100 μl of either rabbit anti-dog IgG-Alexa Fluor 647® conjugate (Jackson ImmunoResearch Laboratories, PA, USA; diluted 1:200 in PBS plus 1% BSA, or with a solution of goat anti-dog IgA-FITC conjugate (Lifespan Biosciences, MA, USA, also diluted 1:200 in PBS plus 1% BSA, and incubated for 30 minutes on ice. The suspensions were then washed twice, and then fixed for 10 minutes in a solution of 4% paraformaldehyde (PFA). After washing, the bacteria were resuspended in 380 μl of PBS, plus 20 μl propidium iodide solution (PI; 1 g/ml; Sigma-Aldrich, St. Louis, Mo., USA), which was added to each sample before flow cytometry analysis.

For detection of serum IgG antibody specific to intestinal bacteria, 6 stock cultures of E. coli isolated from the stool of healthy dogs (n=3) and dogs with IBD (n=3) were generated as described below. The bacteria in short term cultures were collected for detection of IgG binding, using serum from healthy dogs and dogs with IBD followed the previous study method. Briefly, each test serum sample was diluted 1:200 in PBS plus 1% BSA, then added to Escherichia coli in suspension and incubated 30 minutes on ice, then washed followed by the rabbit anti-dog IgG-Alexa Fluor 647® conjugate, and analyzed by flow cytometry, as described previously.

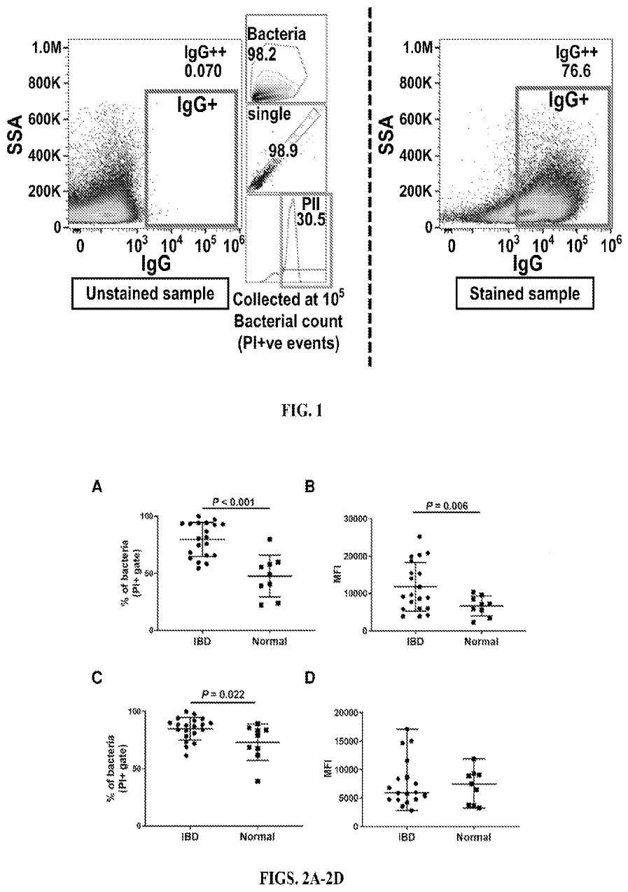

Flow cytometric analysis of fecal samples for IgG and IgA binding was performed using a Beckman Coulter Gallios flow cytometer (Brea, Calif., USA). Analysis was done on 100,000 PI-positive events (PI staining was done to include bacteria (DNA⁺) for analysis and exclude debris without nuclear material (DNA⁻) from analysis). Flow cytometry data was analyzed using FlowJo Software (Ashland, Oreg., USA). The analysis included the percentage of positive fluorescent cells as well as the fluorescence intensity of IgG⁺ or IgA⁺ cells. Background fluorescence levels were determined using bacteria without addition of anti-IgG or IgA antibodies. An example of the typical gating scheme is provided in FIG. 1.

Isolation of E. coli Intestinal Strains and Evaluation of Anti-Bacterial Antibodies Present in Serum.

Six different isolates of E coli, 3 obtained from feces of healthy dogs and 3 from dogs with IBD, were prepared to assess the presence of anti-bacterial antibodies in serum of healthy dogs and dogs with IBD. To isolate E coli, fresh fecal samples of IBD dogs and healthy dogs were collected, homogenized and diluted in phosphate buffered saline (PBS). The fecal suspension was cultured in Tryptic Soy Broth (TSB) (BD, Franklin Lake, N.J., USA) at 37° C. overnight with shaking. The overnight cultured media was plated on McConkey agar and incubated in aerobic condition overnight at 37° C. The cultured colonies were examined the next day, and each E. coli-suspected colony was further subcultured onto blood agar as well as McConkey agar in parallel. The next day, the pure cultures were submitted to confirm the E. coli species by evaluation at the CSU-VTH diagnostic lab.

Six E. coli isolates (3 from dogs with IBD and 3 from normal dogs) were used to test for the relative concentrations of anti-bacterial IgG antibodies present in serum of dogs with IBD and healthy dogs. Briefly, each pure E. coli isolate were cultured in aerobic condition overnight at 37° C. with shaking. The pure E. coli cultured suspension was washed with PBS and centrifuged to get a bacterial pellet. The E. coli pellet was resuspended in 100 μl of dog serum dilution and followed the staining protocol as previously described. Briefly, diluted dog serum was incubated with E. coli on ice for 30 minutes, the bacteria were washed twice, and then incubated with anti-dog IgG or IgA secondary antibody for 30 minutes. The bacterial pellets were washed, fixed with 4% paraformaldehyde (PFA) and propidium iodide (PI) was added before flow cytometry analysis.

### Macrophage Isolation and Culture

Macrophages were derived from differentiated monocytes from blood of healthy dogs as described previously, and were used to assess macrophage activation following incubation with fecal bacteria recovered from healthy dogs and from dogs with IBD. Briefly, PBMC were isolated from EDTA-anticoagulated blood samples by Ficoll-density separation, and the PBMC were resuspended in complete medium (DMEM, 1% Penicillin-streptomycin, essential and non-essential amino acid) with 1% FBS and plated at a density 1×10⁶ PBMC/0.5 ml in 48-well polystyrene cell culture plates, incubated for 4 hours at 37° C. After allowing for monocyte adhesion, the non-adherent cells were washed off with PBS and the remaining monocytes were refed with complete medium with 15% FBS, supplemented with 10 ng/ml huM-CSF (Peprotech, Rocky Hill, N.J., USA) and cultured for 7 days. The medium was changed every 2 days and after 7 days in culture, the monocyte-derived macrophages were used for phagocytosis and cytokine assays.

### Macrophage Phagocytosis and Activation Assays

Fecal bacteria (prepared as noted above) from dogs with IBD (n=5), and normal dogs (n=5) were used in macrophage phagocytosis and activation assays. To assess bacterial phagocytosis, numbers of bacteria (note that bacteria used in these assays were non-viable after freezing) added to macrophage cultures were calculated and equalized by first determining bacterial counts using PI-labeled bacteria and calibrating counts using counting beads (Invitrogen, Eugene, Oreg.). Final numbers of bacteria in samples were calculated by comparing the ratio of bead events to bacterial cell events according to the manufacturer datasheet. The fecal bacteria were added to macrophages at MOI ratio of 5 bacteria per 1 macrophage, and bacteria were spun onto macrophages by centrifugation at 2000× G for 10 minutes, then the macrophages were incubated for 2 hours at 37° C. The cultures were then washed to remove non-phagocytosed bacteria and the cells were detached and performed the flow cytometry. The % of PI+ve macrophage and PI abundance in macrophage were analyzed.

To assess macrophage activation by fecal bacteria, macrophages were incubated with bacteria (MOI=5) for 2 hours to allow phagocytosis, then the non-phagocytosed bacteria were removed, and the macrophages cultured for an additional 24 hours. The supernatants were collected to measure cytokines (TNF-α, IL-10) by ELISA. As a positive control for cytokine release and activation, 10 ng/ml LPS was added to parallel cultures of macrophages. These assays were repeated 3 times using blood from 3 different unrelated donor animals to assure reproducibility.

Flow Sorting and 16S rRNA Sequencing

For these studies, 3 populations of bacteria were analyzed for population composition, using 16S sequencing. The 3 populations consisted of total fecal bacteria from dogs with IBD (n=10), fecal bacteria from healthy dogs (n=10), and bacteria with high levels of bound IgG (IgG^{hi} bacteria), obtained from feces of dogs with IBD (n=10) following incubation with anti-dog IgG secondary antibody, and prepared by cell sorting using a BD FACSAria sorter. To enrich for IgG⁺ bacteria, fecal bacteria from dogs with IBD were immunostained as noted above, and the population of IgG^{hi} bacteria (mean fluorescence intensity or MFI greater than normal baseline) was sorted. The reference population for setting sorting gates was comprised of unstained bacteria. The purity of the sorted bacterial population was assessed by flow cytometry and was found to consist of at least 85% IgG^{hi} bacteria.

Bacteria underwent 16S rRNA sequencing following DNA extraction using a Mobio PowerSoil DNA Isolation kit (Qiagen, Valencia, Calif.) according to manufacturer's instructions. Extracted DNA was submitted for 16S rRNA sequencing and analyzed by Novogene Corporation (Chula Vista, Calif.). The 16S rRNA sequencing was performed as reported in a previous study (32). Sequences analysis were performed by Uparse software (Uparse v7.0.1001) (33). For each representative sequence, Mothur software was performed against the SSUrRNA database of SILVA Database (34). For species annotation at each taxonomic rank (Threshold:0.8˜1), OTUs abundance information were normalized using a standard of sequence number corresponding to the sample with the least sequences. Subsequent analysis of alpha diversity and beta diversity were all performed basing on this output normalized data. Alpha diversity was calculated using Shannon diversity index. Beta diversity on both weighted and unweighted unifrac were calculated by QIIME software (Version 1.7.0). PCoA analysis was displayed by WGCNA package, stat packages and ggplot2 package in R software (Version 2.15.3). Metastats was calculated by R software. P-values were calculated by the method of permutation test while q-values were calculated by method of Benjamini and Hochberg False Discovery Rate (35). Anosim, MRPP and Adonis were performed by R software (Vegan package: anosim function, mrpp function and adonis function). AMOVA was calculated by mothur using amova function. T-test and drawing were conducted by R software.

### Statistical Analysis

Data were analyzed using Prism 7 software (GraphPad, San Diego, Calif., USA). The normality of data was initially analyzed using the Shapiro-Wilk normality test. The normally distributed data were shown as mean±standard deviation (SD). Data which were not normally distributed were reported in median (range). Statistical differences between 2 groups were evaluated using the two-tailed unpaired t-test for parametric data and Mann-Whitney test for non-parametric data as indicated in the text. For statistical assessment of serum IgG response, the % IgG binding E. coli were compared using one-way ANOVA. The result from repeated experiments including cytokine production from different PBMC donors was normalized to baseline control before analysis. To analyze the association between Ig-binding bacteria and other variables including disease activity index, linear regression analysis was performed. The Receiver-Operating Characteristic (ROC) curve was used to determine the sensitivity and specificity as diagnostic ability between IBD and normal dogs. In all examples, the statistical significance was set at P<0.050.

### Results

### Breed Characteristics of Study Dogs

The demographic and disease activity index evaluation of 20 dogs with IBD enrolled in the study are shown in the Table 1. The breeds included were Bernese Mountain Dog (n=4), mixed breeds (n=4), Labrador Retriever (n=2), Yorkshire Terrier (n=2), Pug, Rottweiler, Boxer, Cavalier King Charles Spaniel, German Shorthair Pointer, English Bulldog, American Eskimo, Siberian Husky. The breeds in the healthy control group (n=9) included mixed breed animals, Standard Poodle, Cocker Spaniel, Shih Tsu, Nova Scotia Duck Tolling Retriever, English Coonhound, Chihuahua, English Setter. On average, the disease duration in dogs with IBD was classified as chronic, with moderate disease activity index (Table 1). Endoscopic scores and WSAVA histopathology scores are reported in Table 1. There was no significant difference in age between healthy control dogs and dogs with IBD (P=0.42).

TABLE 1
Demographic data of study groups. 20 IBD dogs and 9 healthy
dogs enrolled in the study. Also, the disease activity
index and other parameters were shown. CIBDAI; Canine
Inflammatory Bowel Disease Activity Index, CCECAI; Canine
Chronic Enteropathy Clinical Activity Index.

| | IBD | Normal |
|---|---|---|
| Sample size | 20 | 9 |
| Gender | | |
| Male | 14 | 4 |
| Female | 6 | 5 |
| Age (year) | 6.4 ± 3.77 | 7.6 ± 3.05 |
| Weight (kg) | 24.04 ± 13.79 | 22.73 ± 11.35 |
| Disease Duration (month) | 4.62 ± 5.27 | — |
| Disease activity index | | NP |
| CIBDAI | 6.7 ± 3.57 | |
| CCECAI | 8.1 ± 4.55 | |
| Endoscopic lesion | 100% | NP |
| Endoscopic Score | | NP |
| Gastroscopic | 1 (0-2) | |
| Duodenoscopic | 3 (2-6) | |
| Ileoscopic | 3 (1-7) | |
| WSAVA Histopathology score | 5.15 ± 2.68 | NP |

Reported as mean ± SD and median (range)
NP = not performed

IgG Binding to Fecal Bacteria in Dogs with IBD Versus Healthy Dogs

The percentage of IgG⁺ bacteria in the feces of dogs with IBD was significantly greater than in feces of healthy control animals: (IBD: 80%±15.05; healthy: 47.5%±18.35, P<0.0001, (FIG. 2A). In addition, the overall amount of IgG bound by bacteria, as assessed by MFI, was significantly higher in dogs with IBD than in healthy dogs (MFI-IBD: 11,769±6,539 a.u.; MFI-healthy: 6,650±2,687 a.u., P=0.005, FIG. 2B).

The percentage of IgA⁺ bacteria was also significantly higher in dogs with IBD than in healthy dogs, though the magnitude of the difference was less than for IgG binding (IBD: 84.86%±9.87; healthy: 73.18%±15.83, P=0.022, FIG. 2C). However, the total amount of IgA bound to bacteria was not significantly different for the two groups of dogs (MFI-IBD: 7,607 (2,834-17,120) a.u.; MFI-healthy: 7,113 (3,280-11,925) a.u., P=0.91, FIG. 2D).

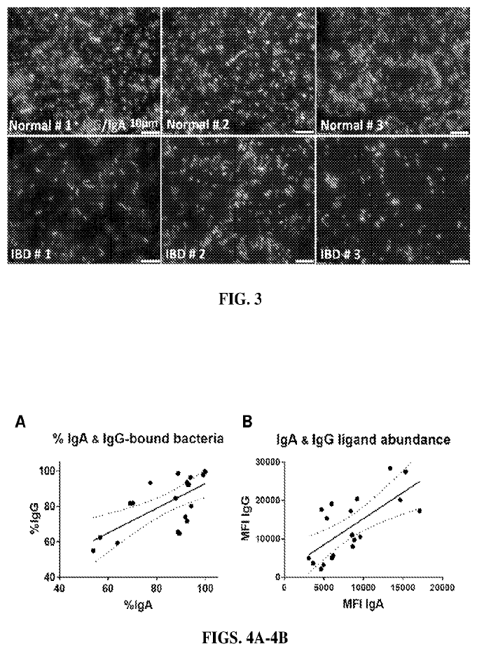

Confocal microscopy was used to visualize the IgG⁺ and IgA⁺ populations of bacteria, and the overlap in the two populations of Ig⁺ bacteria, in dogs with IBD and healthy dogs (FIG. 3). The IgG⁺ bacteria were visualized with rabbit anti-dog IgG-Cy3 conjugate (red) and the IgA⁺ bacteria were visualized with goat anti-dog IgA-FITC conjugate (green), and dual positive bacteria appeared yellow in merged images. In feces of healthy dogs, there was a predominant population of IgA⁺ bacteria, with substantially fewer IgG⁺ bacteria (FIG. 3). In feces from dogs with IBD, many more IgG⁺ bacteria were present, as reflected by the large number of dual positive (yellow) bacteria visualized. Linear regression analysis revealed that there was significant correlation between the percentages of IgG⁺ and IgA⁺ bacteria in dogs with IBD (R²=0.45, P=0.001; FIGS. 4A-4B). A similar correlation of MFI of IgG⁺ and IgA⁺ bacteria was also noted (R²=0.48, P=0.001), showing that increased in IgG binding activity was associated with increased IgA binding in terms of both percentages of bacteria bound and in the abundance of IgG and IgA present on the surface of bacteria. In the case of healthy dogs, there was not a significant correlation between IgG and IgA-binding bacteria.

### Recognition of Fecal Bacteria by Circulating IgG

To determine whether the IgG bound to gut bacteria was produced primarily in the GI tract or was instead produced in extra-intestinal lymphoid tissues and then secondarily transported to the GI tract (eg, by leakage from intestinal vasculature), serum from the presence of IgG antibodies specific for a common intestinal bacterium (E. coli) was assessed. These assays were done using pure cultures of E. coli obtained from the GI tract of dogs to avoid the confounding effects of IgG already present on the surface of GI bacteria obtained directly from feces. To address the possibility that different E. coli strains may exist in the feces of heathy dogs compared to dogs with IBD, different E coli isolates (n=3 each) from healthy dogs and dogs with IBD were also tested.

The amount of IgG present in serum that bound to E coli was not different between healthy dogs and dogs with IBD (FIG. 5). Nor were there differences in serum IgG recognition of E. coli isolated from healthy dogs or from dogs with IBD. Similarly, differences in serum IgA recognition of E coli were not observed in healthy dogs versus dogs with IBD. Thus, the IgG bound to the surface of fecal bacteria was primarily produced locally in the GI tract, rather than being produced systemically. These findings are also consistent with the increased numbers of plasma cells detected in the GI tract of dogs with IBD.

Macrophage Phagocytosis of Fecal Bacteria Increased in Dogs with IBD

Given the presence of significantly more IgG⁺ bacteria in the GI tract of dogs with IBD, links between this phenomenon and induction of intestinal inflammation were analyzed. One plausible mechanism linking IgG⁺ bacteria to GI inflammation involves an interaction of gut bacteria with phagocytic cells such as macrophages. Therefore, an in vitro system was used to determine whether gut bacteria from dogs with IBD were inherently more inflammatory than gut bacteria from healthy dogs.

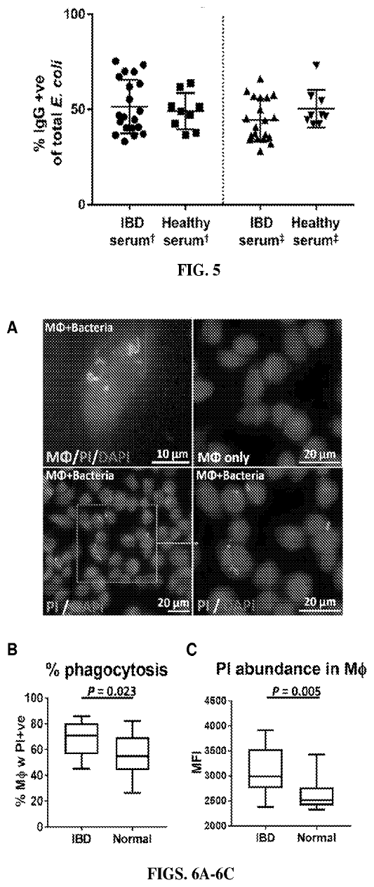

First, the relative ability of dog macrophages to phagocytose bacteria from IBD dogs versus bacteria from healthy dogs was compared (FIGS. 6A-6C). Macrophage phagocytosis of bacteria from IBD dogs was found to be significantly greater than phagocytosis of bacteria from healthy dogs. For example, the percentage of macrophages containing phagocytosed bacteria was 67.91±13.68% in cultures incubated with bacteria obtained from IBD animals, compared to 55.05±15.48% for bacteria from healthy dogs (P=0.023, FIG. 6B). Also, the average numbers of ingested bacteria per individual macrophage (as reflected by increased MFI) was significantly increased in macrophages incubated with bacteria from dogs with IBD [MFI: 2,994 (2,378-3,912)] compared to macrophages fed bacteria from healthy dogs [MFI: 2,519 (2,323-3,428), P=0.005, FIG. 6C]. Thus, GI bacteria in dogs with IBD were more likely to be phagocytosed by macrophages than bacteria from healthy dogs.

Ingestion of Bacteria from Dogs with IBD Triggers Greater Macrophage Inflammatory Response

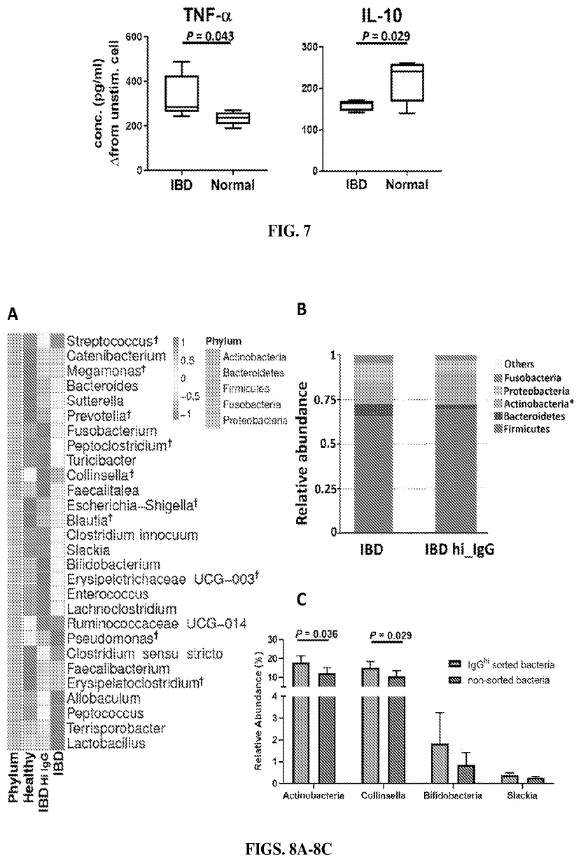

Ingestion of bacteria, particularly via Fc receptor mediated internalization, serves as a strong activating stimulus for macrophages. Next, the impact of fecal bacteria ingestion on macrophage activation and cytokine production was examined Macrophages incubated with bacteria from IBD dogs produced significantly greater amounts of TNF-α than macrophages incubated with bacteria from healthy dogs (FIG. 7). Conversely, macrophages incubated with IBD bacteria produced significantly less IL-10 than macrophages incubated with healthy dog bacteria (FIG. 7). Thus, it was concluded that bacteria present in the gut of dogs with IBD were inherently more immune stimulatory and capable of triggering macrophage production of pro-inflammatory cytokines such as TNF-α, than bacteria from the gut of healthy dogs.

### Microbiome Analysis and Selectivity of IgG Binding

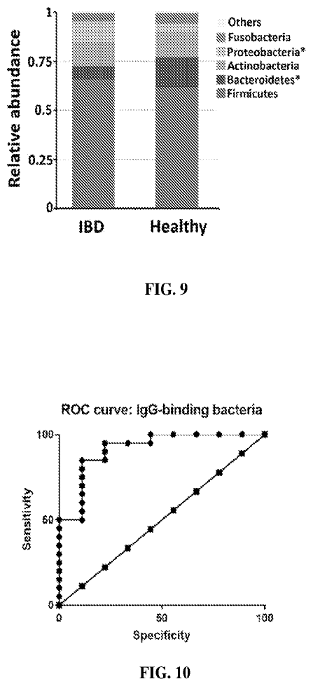

The overall composition and complexity of the GI microbiome in dogs with IBD (n=10) was compared to that of healthy dogs (n=10), using 16S rRNA metagenomics sequencing (FIGS. 8A-8C). Dogs with IBD had a greater abundance of bacteria in the Proteobacteria phylum (P=0.045; FIG. 9) including Escherichia-Shigella and other genera such as Clostridium, Blautia, Bifidobacterium, Enterococcus, Pseudomonas, Faecalibacterium, Lactobacillus, along with a decrease in abundance of Bacteroidetes phyla (P=0.02), and other genera; Streptococcus, Fusobacterium, Peptoclostridium, and Turicibacter, compared to the flora present in healthy control dogs (FIG. 8A). These results are largely in agreement with previous studies of the microbiome in dogs with IBD and show that the current study populations were similar to those of other studies with respect to bacterial diversity and differences in IBD versus healthy dog microbiomes.

Next, 16S sequencing studies were conducted to determine whether bacteria with high levels of IgG binding present in feces of dog with IBD represented uniquely enriched subsets of bacteria (eg, pathogenic bacteria), or whether the IgG^{hi} population of bacteria were evenly distributed amongst all the major phyla (ie, no enrichment for specific phyla or genera).

Bacteria with the highest levels of IgG binding included Collinsella, Faecalitalea, Escherichia-Shigella, Blautia, Bifidobacterium, Clostridium innocuum, Slackia and Enterococcus

TABLE 2
IgG^{hi}-sorted bacteria abundance. Table reported the comparison of
% relative abundance between IgG^{hi}-sorted and non-sorted bacteria
from IBD group. Data shown in Mean ± SD (if parametric data)
and Median (range) (if non-parametric data). The appropriate statistical
comparison of 2 groups either paired t-test and Mann-Whitney test
was performed corresponding the type of data. P value of 0.05 is set.

| | IBD | | | | |
|---|---|---|---|---|---|
| | IgG^{hi} bacteria | | Non-sorted bacteria | | |
| Taxa | (n = 10) | | (n = 10) | | P value |
| Actinobacteria | 17.95 ± 11.08 | | 12.23 ± 9.363 | | 0.036 |
| Bifidobacteria | 0.3 | (14.4) | 0.25 | (5.8) | 0.094 |
| Slackia | 0.38 ± 0.36 | | 0.26 ± 0.21 | | 0.058 |
| Collinsella | 15.1 ± 10.66 | | 10.5 ± 9.75 | | 0.029 |
| Bacteroidetes | 2.38 ± 1.82 | | 6.64 ± 4.07 | | 0.071 |
| Bacteroides | 1.08 ± 0.77 | | 2.43 ± 2.53 | | 0.572 |
| Prevotella | 0.34 | (2.74) | 1.19 | (7.56) | 0.131 |
| Firmicutes | 69.64 ± 10.7 | | 65.99 ± 12.85 | | 0.156 |
| Lachnoclostridium | 0.3 | (0.3) | 0.2 | (0.2) | 0.176 |
| Megamonas | 0.05 | (0.78) | 0.06 | (1.05) | 0.77 |
| Faecalitalia | 0.3 | (0.6) | 0.2 | (0.4) | 0.093 |
| Catenibacterium | 0.6 | (3.8) | 0.75 | (7.8) | 0.477 |
| Clostridium sensu stricto | 0.65 | (2.8) | 0.7 | (6.5) | 0.3 |
| Blautia | 10.93 | (27.03) | 9.65 | (21.52) | 0.492 |
| Enterococcus | 1.4 | (12.2) | 0.8 | (4.8) | 0.089 |
| Streptococcus | 1.17 | (19.69) | 0.75 | (13.74) | 0.275 |
| Clostridium innoculum | 0.15 | (0.8) | 0.1 | (0.5) | 0.140 |
| Lactobacillus | 0.9 | (4.7) | 1.35 | (15.1) | 0.348 |
| Erysipelotrichaceae | 0.14 | (18.8) | 0.45 | (8.43) | 0.002 |
| Turicibacter | 0 | (0.1) | 0.1 | (0.4) | 0.008 |
| Peptoclostridium | 8.0 ± 4.19 | | 10.26 ± 6.78 | | 0.313 |
| Erysipelatoclostridium | 1.96 | (20.78) | 1.63 | (26.47) | 0.921 |
| Faecalibacterium | 0.8 | (31.91) | 0.7 | (31.87) | 0.766 |
| Fusobacteria | 2.77 ± 2.33 | | 4.41 ± 3.03 | | 0.215 |
| Fusobacterium | 2.68 ± 2.31 | | 4.25 ± 2.94 | | 0.223 |
| Proteobacteria | 7.04 ± 6.81 | | 10.61 ± 9.9 | | 0.267 |
| Escherichia-Shigella | 2.67 | (21.75) | 1.57 | (13.76) | 0.557 |
| Sutterella | 0.05 | (0.3) | 0.1 | (0.2) | 0.766 |
| Pseudomonas | 0.05 | (3.9) | 1.18 | (31.61) | 0.084 |

(FIG. 8A and Table 2). The taxa with the lowest levels of IgG binding included Pseudomonas, Clostridium (sensu stricto) and Lactobacillus. There was significant enrichment of bacteria in the Actinobacteria phylum (P=0.036) in the IgG^{hi} population, compared to non-sorted IBD bacteria (FIG. 8C and Table 2). Also, the most abundant genus in the IgG^{hi} Actinobacteria phylum was Collinsella, which was significantly enriched in the IgG^{hi} sorted population of bacteria compared to non-sorted bacteria. Thus, there was preferential immune recognition of Actinobacteria in dogs with IBD.

### Sensitivity and Specificity of Fecal Bacteria IgG Assay for Detection of IBD in Dogs.

The preceding results showed that quantitation of the relative degree of IgG binding to fecal bacteria is useful as a diagnostic test for detection of IBD in dogs. Therefore, the sensitivity and specificity of the flow cytometric assay were evaluated, by assessing either percentage IgG⁺ bacteria, or amount of IgG bound per bacterial cell, for sensitivity and specificity for detecting IBD in dogs. Using receiver operating curves (ROC) (FIG. 10), it was shown that the area under the curve (AUC) for IgG⁺ bacteria was 0.92 (95% CI: 0.80-1.03, P<0.0001). This result showed high diagnostic utility for the flow cytometric test using percentage IgG⁺ bacteria for differentiating IBD dogs from healthy dogs.

Next, the sensitivity and specificity of the bacterial IgG assay were evaluated, using a cutoff point based on the upper limit of 95% confidence interval determined using bacteria from healthy dogs, which was defined as 60% IgG⁺ bacteria. The bacterial percentage IgG⁺ assay was found to have 85% sensitivity (95% CI: 62.11-96.79) and 89% specificity (95% CI: 51.75-99.72) for detection of clinically apparent IBD in dogs. Overall, the fecal IgG test in dogs had a positive likelihood ratio of 7.7 and a negative likelihood ratio of 0.17. However, the percentage of IgG⁺ bacteria was found to not correlate with the disease activity index, including CIBDAI (P=0.71) and CCECAI (P=0.55). In addition, the overall histopathologic score and endoscopic lesion scores did not correlate with the percentage of IgG⁺ bacteria, as determined by linear regression analysis. Thus, the assessment of IgG bound to the surface of bacteria was found to be a very sensitive and specific test for detection of IBD in dogs, though test positivity did not correlate with disease activity or severity. Thus, the fecal IgG assay was considered to have higher sensitivity and specificity for IBD detection than other currently available assays, including fecal calprotectin (S100A12), which was found to be 65% sensitive and 84% specific for diagnosing IBD in dogs.

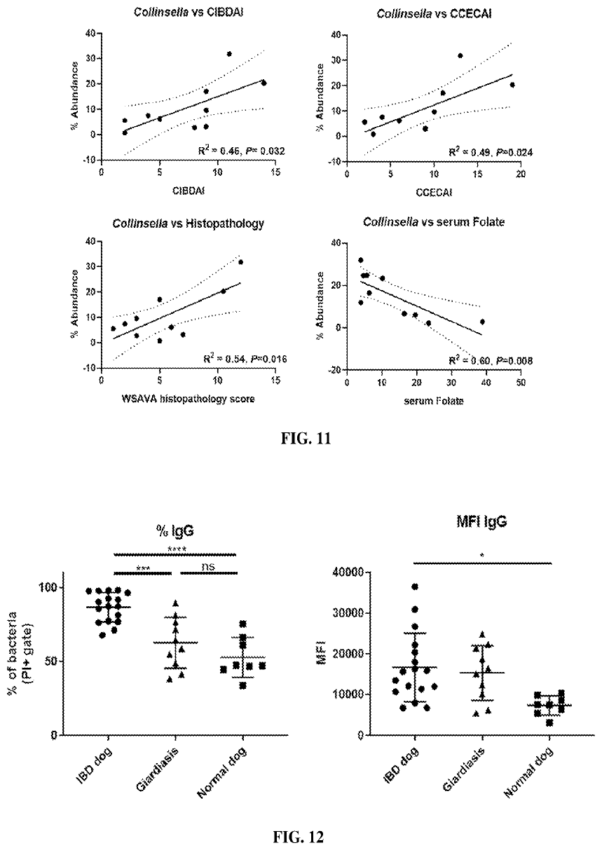

Furthermore, the bacterial IgG assay was performed using fecal bacteria from IBD dogs, dogs having giardiasis, and normal dog controls. The data in FIG. 12 show that the levels of IgG binding to fecal bacteria in dogs with diarrhea due to Giardia infection were significantly lower than in IBD dogs while not different than in normal dogs. These data indicate the test can distinguish two different diarrheal diseases (IBD from giardiasis) and therefore demonstrates the specificity of the assay for IBD in dogs.

### Discussion

The interaction between the host immune response and gut bacteria is now considered a primary driver of intestinal inflammation in humans with IBD. There is evidence of an increase in IgG responses directed against gut bacteria in patients with Crohn's disease. However, a similar phenomenon has not been observed previously in dogs with IBD, and this study is the first to document an immune response against gut bacteria in this disease.

A key finding disclosed herein was that dogs with IBD had significantly higher binding of IgG to gut bacteria, compared to healthy dogs (FIGS. 2A-2D). For example, there were overall 30% more bacteria with surface bound IgG in dogs with IBD than in normal dogs (FIG. 2A). Moreover, these results also show that the source of the anti-bacterial IgG production was from local immunoglobulin production in the gut, rather than from immunoglobulin produced in extra-intestinal sites.

An important difference between immunoglobulin responses to intestinal bacteria in the data disclosed herein and in the data from Crohn's patients is that no increase in IgA binding to bacteria in dogs with IBD was observed, whereas in humans there was significantly more IgA present on gut bacteria compared to gut bacteria in healthy patients. The reasons for this difference between species are not currently known, though it should be noted that soluble IgA concentrations have been shown to be lower overall in the feces of dogs with IBD than in healthy dogs.

These data also demonstrate a link between bacterial IgG binding and induction of intestinal inflammation, which can involve activation of macrophages in the gut. For example, incubation of macrophages with fecal bacteria from dogs with IBD triggered significantly greater macrophage activation and TNF-α production than did bacteria from healthy dog GI tracts (see FIGS. 6A-6C, and 7). Conversely, bacteria from IBD dogs triggered significantly less IL-10 production by macrophages than bacteria from healthy dogs. Thus, the net effect of the interaction of macrophages with IgG bound bacteria in animals with IBD is to trigger greater local immune activation and inflammation. This effect is in part mediated by the interaction of bacterial bound IgG with activating Fc receptors expressed by macrophages in the intestinal epithelium or lamina propria.

Interestingly, the IgG response in dogs with IBD appeared to be directed preferentially towards bacteria considered part of the dysbiotic flora present in the gut. Bacteria in the genus Collinsella had the highest levels of IgG binding in dogs with IBD, whereas this organism was not present in greater abundance in the gut of healthy dogs (see FIG. 8A and Table 1). This genus was noted in the gut microbiome in dogs with gastric-dilation and volvulus as well as reported to be one of the high IgA binding bacteria detected in patients with CD. While not all dysbiotic flora are pathogenic, certainly some of the genera represented in the dysbiotic gut (eg, Escherichia, Clostridium and Enterococcus) have been associated with intestinal infection and invasion. These pathogenic bacteria, particularly if enteroinvasive or capable of enhanced GI colonization, can trigger greater immune recognition and local antibody production.

In addition, there is no association between the level of IgG⁺ bacteria and clinical parameters associated with disease activity index, histopathology score, or endoscopic score. Like previous IBD studies in dogs, clinical parameters showed no correlation or a weak correlation with other IBD parameters including serum Ig, C-reactive protein and Calprotectin. However, the presence of bacteria in the genus Collinsella, which was found with the highest IgG binding in dogs with IBD (see FIG. 8C), showed the highest association with common IBD clinical parameters including CIBDAI (P=0.032), CCECAI (P=0.024), histopathology scores (P=0.016) and serum folate concentrations (P=0.008, see FIG. 11) in agreement with previous studies in humans and in cats. In humans, Collinsella is considered as one of the taxa used to discriminate between patients with UC and CD.

Using a culture-independent approach of 16S rRNA metagenomic sequencing for microbiome analysis, the data herein showed relatively good agreement with prior sequencing studies of IBD in dogs, analyzing either fecal or mucosa-associated microbiota. For example, one prior study found dysbiosis of commensal bacteria including increased Proteobacteria (e.g., E. coli), Clostridium, and Enterococcus. In this example, it was shown that these expanded populations of dysbiotic bacteria have high levels of IgG bound on their surface (FIG. 8A) resulting in increased overall percentage and higher overall MFI of IgG-binding bacteria in dogs with IBD (see FIGS. 2A-2D). However, some of the highest IgG binding taxa identified in this study were considered as non-IBD associated taxa in previous studies in dogs with IBD, including Faecalibacterium, Allobaculum, Slackia and Clostridium.

It is important to note that some of the dogs with IBD had received antibiotic therapy prior to their enrollment in this study, inasmuch as antibiotic treatment is known to significantly alter the intestinal microbiome in dogs. However, no significant difference in the overall percentages of IgG⁺ bacteria in dogs was found, regardless of their antibiotic pre-treatment status, showing that antibiotic treatment had little discernable effect on generation of anti-bacterial IgG in dogs with IBD. This is an important observation because it shows that the fecal IgG assay is relatively resistant to interference by prior antimicrobial therapy.

In summary, a high percentage of intestinal bacteria are recognized by IgG produced locally in the gut in dogs with IBD, and IgG bound bacteria are linked to intestinal inflammation. In terms of diagnostic utility, the data herein demonstrate that the use of a bacterial flow cytometric IgG assay provided significant sensitivity and specificity for differentiating dogs with IBD from healthy dogs, using fecal samples (FIG. 10). Since there are currently no commercially available assays for accurately identifying dogs with IBD, this bacterial flow cytometric assay provides a useful clinical test that can be run on fresh or frozen fecal samples.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method for treating inflammatory bowel disease (IBD) in a companion animal, comprising:
collecting a fecal sample from the companion animal;
incubating the fecal sample with a detecting antibody which specifically binds to an IgG antibody;
determining a proportion of fecal bacteria in the fecal sample that are bound by IgG antibody;
diagnosing that the animal is susceptible to or suffering from IBD when the proportion of fecal bacteria in the fecal sample that are bound by the IgG antibody is greater than about 60%; and
if the animal is susceptible to or suffering from IBD:
i) administering to the companion animal an effective amount of a therapeutic agent for treating the IBD,
ii) changing the diet of the companion animal, or
iii) performing a fecal transfaunation.

2. The method of claim 1, wherein the detecting antibody is conjugated to a fluorescent moiety.

3. The method of claim 1, wherein the proportion of fecal bacteria in the fecal sample that are bound by the IgG antibody is determined by flow cytometry.

4. The method of claim 1, wherein the companion animal is a canine.

5. The method of claim 4, wherein the companion animal is a dog.

6. The method of any one of claim 1, wherein the companion animal is a feline.

7. The method of claim 6, wherein the companion animal is a cat.

8. The method of any one of claim 1, wherein the therapeutic agent is selected from an antibiotic, an immunosuppressive agent, or a probiotic.

9. The method of claim 8, wherein the antibiotic comprises metronidazole, tylosin, or ampicillin.

10. The method of claim 8, wherein the immunosuppressive agent comprises prednisone, prednisolone, budesonide, cyclosporine, mycophenolate, or chlorambucil.

11. A method for diagnosing inflammatory bowel disease (IBD) in a companion animal, comprising:
collecting a fecal sample from the companion animal;
incubating the fecal sample with a detecting antibody which specifically binds to an IgG antibody;
determining a proportion of fecal bacteria in the fecal sample that are bound by IgG antibody; and
diagnosing that the animal is susceptible to or suffering from IBD when the proportion of fecal bacteria in the fecal sample that are bound by the IgG antibody is greater than about 60%.

12. The method of claim 11, wherein the method further comprises administering to the companion animal an effective amount of a therapeutic agent for treating the IBD, changing the diet of the companion animal, or performing a fecal transfaunation, if the animal is susceptible to or suffering from IBD.

13. The method of claim 11, wherein the detecting antibody is conjugated to a fluorescent moiety.

14. The method of claim 11, wherein the proportion of fecal bacteria in the fecal sample that are bound by IgG antibody is determined by flow cytometry.

15. The method of claim 11, wherein the companion animal is a canine.

16. The method of claim 15, wherein the companion animal is a dog.

17. The method of claim 11, wherein the companion animal is a feline.

18. The method of claim 17, wherein the companion animal is a cat.

19. The method of claim 11, wherein the therapeutic agent is selected from an antibiotic, an immunosuppressive agent, or a probiotic.

20. The method of claim 19, wherein the antibiotic comprises metronidazole, tylosin, or ampicillin.

21. The method of claim 20, wherein the immunosuppressive agent comprises prednisone, prednisolone, budesonide, cyclosporine, mycophenolate, or chlorambucil.
